(19) United States
(12) Patent Application Publication
(10) Pub. No.: US 2025/0268812 A1
(43) Pub. Date: Aug. 28, 2025

(54) COSMETIC COMPOSITION FOR SKIN IMPROVEMENT

(71) Applicants: LG H&H CO., LTD., Seoul (KR); DURAE CORPORATION, Anyang-si (KR)

(72) Inventors: Gwang Jin LEE, Seoul (KR); Hye In WOO, Seoul (KR); Hye Jin LEE, Seoul (KR); Ji Won PARK, Seoul (KR); Soo Yeon KIM, Seoul (KR); Hyeon Jun JEON, Gunpo-si (KR)

(73) Assignees: LG H&H CO., LTD., Seoul (KR); DURAE CORPORATION, Anyang-si (KR)

(21) Appl. No.: 19/052,472

(22) Filed: Feb. 13, 2025

(30) Foreign Application Priority Data
Feb. 28, 2024 (KR) 10-2024-0028971
Nov. 15, 2024 (KR) 10-2024-0163331

(51) Int. Cl. A61K 8/9789; A61K 8/44; A61K 8/49; A61K 8/9728; A61K 8/9794; A61Q 19/08

(52) U.S. Cl. CPC A61K 8/9789; A61K 8/44; A61K 8/445; A61K 8/4913; A61K 8/4946; A61K 8/9728; A61K 8/9794; A61Q 19/08

(57) ABSTRACT

The present invention relates to a cosmetic composition for skin improvement, and more particularly, to a cosmetic composition that solves the odor problem of herbal extracts and is effective in preventing and reducing the loss of skin elasticity and the formation of wrinkles, which are aspects of skin aging.

### CROSS-REFERENCE TO RELATED APPLICATION

This application claims priority to and the benefit of Korean Patent Application No. 10-2024-0163331, filed on Nov. 15, 2024 and Korean Patent Application No. 10-2024-0028971, filed on Feb. 28, 2024, the disclosures of which are incorporated herein by reference in their entirety.

### BACKGROUND

### 1. Field of the Invention

The present invention relates to a cosmetic composition for skin improvement, and more particularly, to a cosmetic composition that solves the odor problem of herbal extracts and is effective in preventing and reducing the loss of skin elasticity and the formation of wrinkles, which are aspects of skin aging.

Gongjindan, made from rare and expensive medicinal herbs, has been a royal tonic that was presented to the Chinese imperial court to protect the health of emperors and empresses. It is based on the five major family prescriptions of Wei Yilin, the greatest physician of the Yuan Dynasty, and was recorded in the medical book “Shiyi Dexiaofang” as a tonic prescription presented to the emperor. As described in the medical book, the efficacy of Gongjindan lies in “water rising and fire descending.” This means it regulates the fundamental energies of the body, water and fire, strengthening innate vitality by raising water energy and lowering fire energy. Among the tonics for longevity including Gongjindan, Gyeongokgo, Bulrowan, Yeonsudan, and Boryeong Yeonmyeongdan, Gongjindan is considered the supreme medicine. Typically, oriental herbal cosmetics including mixed extracts of plants and herbal medicines, which are inspired by tonic prescriptions like Gongjindan, achieve skin improvement effects by using the mixture, but face problems such as odor, composition stability, and user satisfaction. Considering these problems, there are limitations on increasing the mixing ratio to enhance skin improvement effects.

Against this background, the inventors of the present invention made efforts to solve the conventional problems and confirmed the possibility of deriving ingredients for enhancing elasticity and reducing wrinkles through research on the components and efficacy of five medicinal herbs. Accordingly, the inventors confirmed that it is possible to increase the effects of enhancing elasticity and reducing wrinkles and solve formulation problems caused by the limitations of extract mixing by combining the derived glycoprotein fraction and two types of amino acids, and completed the present invention.

### SUMMARY OF THE INVENTION

The present invention is directed to providing a cosmetic composition including a glycoprotein plant extract of Angelica gigas root, Cornus officinalis fruit, Thymus vulgaris, Ganoderma lucidum, and Asparagus cochinchinensis root as an active ingredient.

The present invention is also directed to providing a cosmetic composition including, as an active ingredient, a glycoprotein plant extract of Angelica gigas root, Cornus officinalis fruit, Thymus vulgaris, Ganoderma lucidum, and Asparagus cochinchinensis root, and further including, as an active ingredient, a plant extract of Angelica gigas root, Cornus officinalis fruit, Thymus vulgaris, Ganoderma lucidum, and Asparagus cochinchinensis root.

The present invention is also directed to providing an external skin preparation for wrinkle reduction, including, as an active ingredient, a plant extract of Angelica gigas root, Cornus officinalis fruit, Thymus vulgaris, Ganoderma lucidum, and Asparagus cochinchinensis root, and further including, as an active ingredient, a glycoprotein plant extract of Angelica gigas root, Cornus officinalis fruit, Thymus vulgaris, Ganoderma lucidum, and Asparagus cochinchinensis root.

These are explained in detail as follows. Each description and embodiment disclosed in the present invention may also be applied to other descriptions and embodiments. That is, all combinations of the various elements disclosed in the present invention are included in the scope of the present invention. In addition, it should not be construed that the scope of the present invention is limited by the specific descriptions provided below.

According to an aspect of the present invention, there is provided a cosmetic composition including a glycoprotein plant extract of Angelica gigas root, Cornus officinalis fruit, Thymus vulgaris, Ganoderma lucidum, and Asparagus cochinchinensis root as an active ingredient.

According to another aspect of the present invention, there is provided a cosmetic composition including, as an active ingredient, a glycoprotein plant extract of Angelica gigas root, Cornus officinalis fruit, Thymus vulgaris, Ganoderma lucidum, and Asparagus cochinchinensis root, and further including, as an active ingredient, a plant extract of Angelica gigas root, Cornus officinalis fruit, Thymus vulgaris, Ganoderma lucidum, and Asparagus cochinchinensis root.

According to still another aspect of the present invention, there is provided an external skin preparation for wrinkle reduction including, as an active ingredient, a plant extract of Angelica gigas root, Cornus officinalis fruit, Thymus vulgaris, Ganoderma lucidum, and Asparagus cochinchinensis root, and further including, as an active ingredient, a glycoprotein plant extract of Angelica gigas root, Cornus officinalis fruit, Thymus vulgaris, Ganoderma lucidum, and Asparagus cochinchinensis root.

Hereinafter, the present invention will be described in more detail.

The term “Angelica gigas root” used in the present invention refers to a biennial or triennial herb that grows in relatively humid soil and is an herbal medicine with an excellent hematopoietic effect and outstanding anti-cancer, antioxidant, and anti-inflammatory properties.

The term “Cornus officinalis fruit” used in the present invention refers to the fruit of the Cornus officinalis tree, which is an herbal medicine with antioxidant and anti-inflammatory effects.

The term “Thymus vulgaris” used in the present invention refers to a perennial plant in the mint family (Lamiaceae), also known as thyme, which is an herb used for its effectiveness in treating headaches and bronchitis, expectorant properties, diuretic effects, and for its antibacterial, preservative, and digestive-promoting properties.

The term “Ganoderma lucidum” used in the present invention refers to the Ganoderma lucidum mushroom, a medicinal plant with excellent antioxidant activity that eliminates harmful free radicals in the body.

The term “Asparagus cochinchinensis root” used in the present invention refers to the root of a perennial plant in the Asparagus family, which is an herbal medicine with pharmacological effects including anti-asthmatic, anti-inflammatory, antioxidant properties, alleviation of Alzheimer's disease symptoms, neuroprotection, and promotion of intestinal health.

The term “glycoprotein” used in the present invention refers to a protein with carbohydrates attached thereto. Specifically, it refers to a protein where sugar chains are covalently bonded to amino acids, and the process of adding sugar chains to a protein is called glycosylation. Glycoproteins are defined as proteins in which polypeptides are combined with glycans.

The term “glycoprotein plant extract” used in the present invention refers to a glycoprotein plant extract including Angelica gigas root, Cornus officinalis fruit, Thymus vulgaris, Asparagus cochinchinensis root, and Ganoderma lucidum as active ingredients.

The glycoprotein plant extract according to the present invention promotes the expression of elasticity-related factors such as COL1A1, fibrillin elasticity factors, hyaluronic acid synthesis factors, and the differentiation of adipose-derived stem cells, thereby enhancing skin elasticity, preventing odor development even at high temperatures, and providing stable physical properties and feeling of use for cosmetic compositions, such as reduced stickiness.

The glycoprotein may be obtained by treating the plants with one or more proteases.

The glycoprotein plant extract may contain a sum of aspartic acid, arginine, and glutamic acid that is 30% or more of the total amino acids constituting the glycoprotein.

The cosmetic composition may further include an amino acid.

The term “amino acid” used in the present invention refers to a substance that helps the growth of fibroblasts and keratinocytes, which are the main cells constituting the epidermis and dermis of the skin.

The amino acid may include one or more selected from the group consisting of arginine, histidine, lysine, aspartic acid, serine, threonine, asparagine, glutamine, tyrosine, alanine, isoleucine, leucine, valine, phenylalanine, methionine, tryptophan, glycine, proline, cysteine, and glutamic acid.

Preferably, the amino acid may include arginine or glutamic acid, but is not limited thereto.

The cosmetic composition may further include an amino acid, thereby enhancing skin elasticity, preventing odor development even at high temperatures, and providing stable physical properties and feeling of use for the cosmetic composition, such as reduced stickiness.

The glycoprotein plant extract may be included in an amount of 0.0001 to 10 parts by weight, preferably 0.001 wt % or more, based on 100 parts by weight of the composition.

When the amount of the glycoprotein plant extract is less than 0.0001 parts by weight, the wrinkle reducing effect on the skin may be insignificant, and when the amount of the glycoprotein plant extract exceeds 10 parts by weight, the effect may be ineffective relative to the weight.

The amino acid may be included in an amount of 0.00001 to 10 parts by weight, preferably 0.0001 wt % or more, based on 100 parts by weight of the composition.

When the amount of the amino acid is less than 0.00001 parts by weight, the wrinkle reducing effect on the skin may be insignificant, and when the amount of the amino acid exceeds 10 parts by weight, the effect may be ineffective relative to the weight.

In order to achieve the above objects, another aspect of the present invention provides a cosmetic composition further including a plant extract of Angelica gigas root, Cornus officinalis fruit, Thymus vulgaris, Ganoderma lucidum, and Asparagus cochinchinensis root as an active ingredient in addition to the glycoprotein plant extract as an active ingredient.

The term “plant extract” used in the present invention refers to an extract including Angelica gigas root, Cornus officinalis fruit, Thymus vulgaris, Asparagus cochinchinensis root, and Ganoderma lucidum as active ingredients.

The plant extract may be included in an amount of 0.01 to 30 parts by weight, preferably 0.1 wt % or more, based on 100 parts by weight of the composition.

When the amount of the plant extract is less than 0.01 parts by weight, the wrinkle reducing effect on the skin may be insignificant, and when the amount of the plant extract exceeds 30 parts by weight, the effect may be ineffective relative to the weight.

The cosmetic composition of the present invention may be prepared in any formulation commonly prepared in the art, and it may be formulated as a solution, a suspension, an emulsion, paste, a gel, a cream, a lotion, powder, soap, a cleansing oil, a powder foundation, an emulsion foundation, a wax foundation, a pack, a massage cream, or a spray, but is not limited thereto. More specifically, it may be prepared in the form of a toner, a lotion, a softening lotion, a nourishing lotion, a nourishing cream, a massage cream, an essence, an eye cream, a cleansing cream, a cleansing foam, a cleansing water, a pack, a spray, or powder.

In order to achieve the above objects, still another aspect of the present invention provides an external skin preparation for wrinkle reduction including, as an active ingredient, a plant extract of Angelica gigas root, Cornus officinalis fruit, Thymus vulgaris, Ganoderma lucidum, and Asparagus cochinchinensis root, and further including, as an active ingredient, a glycoprotein plant extract of Angelica gigas root, Cornus officinalis fruit, Thymus vulgaris, Ganoderma lucidum, and Asparagus cochinchinensis root.

The term “external skin preparation” used herein refers to a semi-solid preparation with appropriate consistency for application to the skin, a preparation that is solid at room temperature but softens at body temperature for skin application.

Specifically, the external skin preparation may include external tablets, external solutions, ointments, hard suppositories, and rectal suppositories for external use, and may include personal hygiene products as quasi-drugs. Preferably, it may be a disinfectant cleanser, shower foam, gargle, wet wipes, detergent soap, or hand wash, but is not limited thereto.

The above quasi-drugs may be used together with other quasi-drug ingredients and used appropriately according to conventional methods, and the mixing amount of active ingredients may be determined appropriately according to the purpose of use.

In a specific embodiment of the present invention, it was confirmed that the cosmetic composition and external skin preparation of the present invention enhance skin elasticity and improve odor and physical properties.

Therefore, through the above specific embodiment, it was confirmed that the cosmetic composition of the present invention has the effect of preventing odor development at high temperatures and enhancing skin elasticity, which suggests that it may be used as a technology for improving the physical properties of herbal materials and as an eco-friendly technology.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a graph showing the expression level of the elasticity-related factor ELN after adding the cosmetic composition of the present invention to cells;

FIG. 2 is a graph showing the expression level of the elasticity-related factor COL1A1 after adding the cosmetic composition of the present invention to cells;

FIG. 3 is a photograph showing fibrillin synthesis after UVB irradiation following the addition of the cosmetic composition of the present invention to cells;

FIG. 4 is a graph showing the amount of fibrillin synthesis after UVB irradiation following the addition of the cosmetic composition of the present invention to cells;

FIG. 5 is a graph showing the skin elasticity improvement rate after using the cosmetic composition of the present invention;

FIG. 6 is a graph showing the results of promoting the expression of the elasticity-related factor COL1A1 by the glycoprotein plant extract of the present invention;

FIG. 7 is a graph showing the results of inhibiting the expression of an elasticity-degrading enzyme by the glycoprotein plant extract of the present invention;

FIG. 8 is a set of photographs and graphs showing the results of fibrillin elasticity factor production by the glycoprotein plant extract of the present invention;

FIG. 9 is a graph showing the results of promoting the expression of hyaluronic acid synthesis factors by the glycoprotein plant extract of the present invention;

FIG. 10 is a photograph showing the results of adipose-derived stem cell differentiation by the glycoprotein plant extract of the present invention; and

FIG. 11 is a graph showing the results of lipid accumulation ability of the glycoprotein plant extract of the present invention.

### DETAILED DESCRIPTION OF EXEMPLARY EMBODIMENTS

Hereinafter, the present invention will be described in more detail through examples. These examples are for illustrating the present invention in more detail, and the scope of the present invention is not limited thereby.

### Example 1: Preparation of Complex Plant Extract

30 g each of dried Angelica gigas root, Cornus officinalis fruit, Thymus vulgaris, Asparagus cochinchinensis root, and Ganoderma lucidum were mixed with 900 g of 30% (w/w) butylene glycol and extracted while stirring at 25° C. for 3 days. Each extract was then filtered through a 0.45 m membrane filter, and the filtered extracts were mixed in appropriate ratios to prepare the “complex plant extract.”

### Example 2-1: Preparation of Angelica Gigas Root Glycoprotein Powder

Purified water equivalent to 20 times the weight of dried Angelica gigas root was mixed at a temperature of 50 to 60° C., and a protease was added at a concentration of 0.5% relative to the purified water, and then the mixture was allowed to react for more than 24 hours. The reaction mixture was then heated at a temperature of 90 to 100° C. for 2 hours to inactivate the enzyme and terminate the extraction and enzymatic reaction. Afterward, the extract was cooled, filtered, and concentrated, and glycoprotein powder was obtained by subjecting the concentrated solution to ethanol precipitation. The resulting product was purified in the same manner and dried to complete the final powder.

### Example 2-2: Preparation of Cornus officinalis Fruit Glycoprotein Powder

Purified water equivalent to 20 times the weight of dried Cornus officinalis fruit was mixed at a temperature of 50 to 60° C., and a protease was added at a concentration of 0.5% relative to the purified water, and then the mixture was allowed to react for more than 24 hours. The reaction mixture was then heated at a temperature of 90 to 100° C. for 2 hours to inactivate the enzyme and terminate the extraction and the enzymatic reaction. Afterward, the extract was cooled, filtered, and concentrated, and glycoprotein powder was obtained by subjecting the concentrated solution to ethanol precipitation. The resulting product was purified using the same method and dried to complete the final powder.

### Example 2-3: Preparation of Thymus vulgaris Glycoprotein Powder

Purified water equivalent to 20 times the weight of the dried Thymus vulgaris was mixed at a temperature of 50 to 55° C., and a protease was added at a concentration of 0.5% relative to the purified water, and then the mixture was allowed to react for more than 24 hours. The reaction mixture was then heated at a temperature of 90 to 100° C. for 2 hours to inactivate the enzyme and terminate the extraction and enzymatic reaction. Afterward, the extract was cooled, filtered, and concentrated, and glycoprotein powder was obtained by subjecting the concentrated solution to ethanol precipitation. The resulting product was purified in the same manner and dried to complete the final powder.

### Example 2-4: Preparation of Asparagus cochinchinensis Root Glycoprotein Powder

Purified water equivalent to 20 times the weight of dried Asparagus cochinchinensis root was mixed at a temperature of 50 to 55° C., and a protease was added at a concentration of 0.5% relative to the purified water, and then the mixture was allowed to react for more than 24 hours. The reaction mixture was then heated at a temperature of 90 to 100° C. for 2 hours to inactivate the enzyme and terminate the extraction and enzymatic reaction. Afterward, the extract was cooled, filtered, and concentrated, and the concentrated solution was freeze-dried to obtain glycoprotein powder.

### Example 2-5: Preparation of Ganoderma lucidum Glycoprotein Powder

Purified water equivalent to 20 times the weight of dried Ganoderma lucidum was mixed at a temperature of 50 to 55° C., and a protease was added at a concentration of 0.5% relative to the purified water, and then the mixture was allowed to react for more than 24 hours. The reaction mixture was then heated at a temperature of 90 to 100° C. for 2 hours to inactivate the enzyme and terminate the extraction and enzymatic reaction. Afterward, the extract was treated with activated carbon, cooled, filtered, and concentrated. The concentrated solution was freeze-dried to obtain glycoprotein powder.

### Example 2-6: Preparation of Complex Plant-Derived Glycoprotein Extract

The glycoprotein powders prepared in Examples 2-1 to 2-5 were mixed at a mass ratio of 2-1:2-2:2-3:2-4:2-5, where each mass was selected from a number between 1 and 20. The mixture was then solubilized in purified water and filtered to prepare the “complex plant-derived glycoprotein extract.”

### Comparative Example 1: Preparation of General Complex Plant Extract

Each of the five dried raw materials, including Angelica gigas root, Cornus officinalis fruit, Thymus vulgaris, Asparagus cochinchinensis root, and Ganoderma lucidum, was mixed with purified water at 20 times their weight at a temperature of 50 to 55° C. and extracted for 24 hours. Afterward, each extract was cooled, filtered, and concentrated, and the concentrated solution was freeze-dried to obtain an extract powder. The obtained powders were mixed in the same ratio as in Example 2-6, solubilized in purified water, and then filtered to prepare the “general complex plant extract.”

### Example 3: Preparation of Two Amino Acids

Amino acids, arginine and glutamic acid, were purchased from AJINOMOTO Co., Inc. (Japan), solubilized at a 1:1 molar ratio, repeatedly filtered, and then dried to obtain a powder for use.

### Experimental Example 1: Extract Composition Analysis

The total sugar content, crude protein content, and amino acid composition of the complex plant-derived glycoprotein extract obtained in Example 2-6 and the general complex plant extract obtained in Comparative Example 1 were analyzed and compared to confirm and analyze the glycoprotein in Example 2-6. The results are shown in Tables 1 to 3 below.
Total sugar was measured using the phenol-sulfuric acid method, crude protein was measured using the Lowry method (protein quantification), and amino acid composition was analyzed using liquid chromatography. Glycoprotein confirmation and analysis was performed using the Pierce™ Glycoprotein Carbohydrate Estimation Kit from Thermo Scientific on the powder obtained through protein precipitation using the ammonium sulfate precipitation method and dialysis.

TABLE 1
Total sugar and crude protein analysis

| | Example 2-6 | Comparative |
|---|---|---|
| | (complex | Example 1 |
| | plant-derived | (general complex |
| Units (ppm) | glycoprotein extract) | plant extract) |
| Total sugar | 2219 | 1824 |
| Crude protein | 683 | 398 |

As shown in Table 1, it was confirmed that the complex plant-derived glycoprotein extract of Example 2-6 has 1.22 times higher total sugar content and 1.72 times higher crude protein content compared to the general complex plant extract of Comparative Example 1.

TABLE 2
Amino acid composition ratio (Total = 100%)

| | Example 2-6 | Comparative Example 1 |
|---|---|---|
| Amino | (complex plant-derived | (general complex |
| acid (%) | glycoprotein extract) | plant extract) |
| Aspartic acid | 25.49 | 8.77 |
| Glutamic acid | 20.72 | 14.97 |
| Serine | 4.66 | 4.34 |
| Histidine | 1.49 | 1.83 |
| Glycine | 3.16 | 6.63 |
| Threonine | 4.07 | 5.39 |
| Citrulline | 0.78 | 0.41 |
| Arginine | 17.51 | 2.86 |
| Alanine | 5.22 | 5.88 |
| GABA | 1.31 | 0.08 |
| Tyrosine | 0.81 | 3.76 |
| Valine | 3.08 | 9.42 |
| Phenylalanine | 0.97 | 5.56 |
| Isoleucine | 1.65 | 5.93 |
| Leucine | 2.45 | 9.25 |
| Lysine | 2.53 | 9.02 |
| Proline | 4.09 | 5.88 |

As shown in Table 2, it was confirmed that the complex plant-derived glycoprotein extract of Example 2-6 contains particularly large amounts of aspartic acid, glutamic acid, and arginine in its amino acid composition. The sum of these three amino acids in Example 2-6 was 63.7%, while the sum of these three amino acids in Comparative Example 1 was 26.6%.

TABLE 3
Glycoprotein identification and analysis

| | Treatment | |
|---|---|---|
| | concentration | Absorbance |
| Protein type | (mg/mL) | (550 nm) |
| Blank | 0 | 0.296 |
| Lysozyme | 2.5 | 0.333 |
| Bovine serum albumin (BSA) | 2.5 | 0.413 |
| Ovalbumin | 2.5 | 0.573 |
| Apo-transferrin | 2.5 | 0.798 |
| Fetuin | 2.5 | 5.593 |
| α1-Acid glycoprotein | 2.5 | 5.274 |
| Example 2-6 | 2.5 | 13.148 |
| (complex plant-derived | | |
| glycoprotein extract) | | |

As shown in Table 3, when analyzed at the same concentration, the absorbance of the glycoprotein extract of Example 2-6 was significantly higher than that of known glycoproteins such as fetuin and al-acid glycoprotein. This indicates that the extract contains a large amount of glycoprotein.

### Examples 4 and 5 and Comparative Examples 2 to 4: Preparation of Mixed Composition

The complex plant extract (Example 1), complex plant-derived glycoprotein extract (Example 2-6), and a solution of two amino acids mixed in a specific ratio (Example 3) were prepared, and these were designated as Comparative Examples 2 to 4 and Examples 4 and 5, as shown in Table 4 below.

TABLE 4

| | Com- | Com- | Com- | | |
|---|---|---|---|---|---|
| | parative | parative | parative | Exam- | Exam- |
| Composition | Example 2 | Example 3 | Example 4 | ple 4 | ple 5 |
| Complex plant | O | O | O | O | O |
| extract | | | | | |
| (Example 1) | | | | | |
| Complex plant- | X | X | X | O | O |
| derived | | | | | |
| glycoprotein | | | | | |
| extract | | | | | |
| (Example 2-6) | | | | | |
| Arginine & | X | X | O | X | O |
| Glutamic acid | | | | | |
| (Example 3) | | | | | |
| Total content | 1.0 | 5.0 | 1.0 | 1.0 | 1.0 |
| (wt %) | | | | | |

* The complex plant-derived glycoprotein extract is mixed at an appropriate ratio in the range of 0.001-0.1 wt % based on 100 wt % of the total composition, and the two amino acids are mixed at an appropriate ratio in the range of 0.0001-0.1 wt %.
* The total content is the sum of the contents of each composition in the solution and is the final concentration applied to the following experiments (Experimental Examples 2 to 5).

### Experimental Example 2: Efficacy in Promoting Expression of Elasticity-Enhancing Factors (ELN and COL1A1)

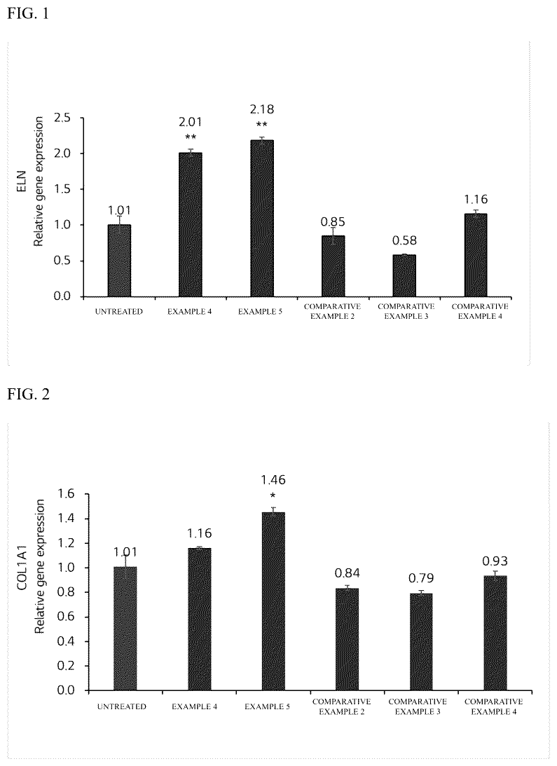

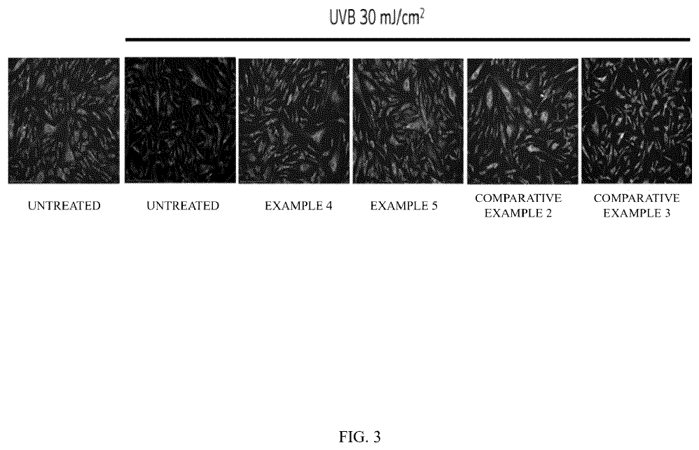

To measure the expression levels of ELN, a gene for elastin protein, and COL1A1, a gene for Type I collagen protein, in cells, human dermal fibroblasts (ATCC) were seeded at 2*10⁵ cells/well in 6-well plates and cultured overnight in a CO₂ incubator. The solutions of Comparative Examples and Examples were added to the culture medium at their final contents and cultured in an incubator for 24 hours. RNA was extracted according to the protocol using the RNA-spin Total RNA Extraction Kit (Intron Biotechnology, Korea), and real-time qPCR was performed using SYBR Green Primer to determine the expression levels of ELN and COL1A1 induced by the solutions of Comparative Examples and Examples (FIGS. 1 and 2).

As shown in FIG. 1, the results showed that for the elastin protein gene ELN, Comparative Examples 2 to 4 did not show an increase in ELN expression compared to the untreated control. However, Examples 4 and 5 showed an increase in ELN expression level of 2.01 and 2.18 times, respectively. In particular, the solution of Example 5, which contained the complex plant extract, complex plant-derived glycoprotein extract, and two amino acids, demonstrated the highest increase in ELN expression, and this result was confirmed to be statistically significant.

As shown in FIG. 2, for the collagen Type I protein gene COL1A1, Comparative Examples 2 to 4 did not show an increase in COL1A1 expression compared to the untreated control, but Examples 4 and 5 showed an increase in COL1A1 expression level of 1.16 and 1.46 times, respectively. In particular, the solution of Example 5, which contained the complex plant extract, complex plant-derived glycoprotein extract, and two amino acids, demonstrated the highest increase in COL1A1 expression, and this result was confirmed to be statistically significant.

### Experimental Example 3: Efficacy in Increasing Elasticity-Related Protein Fibrillin

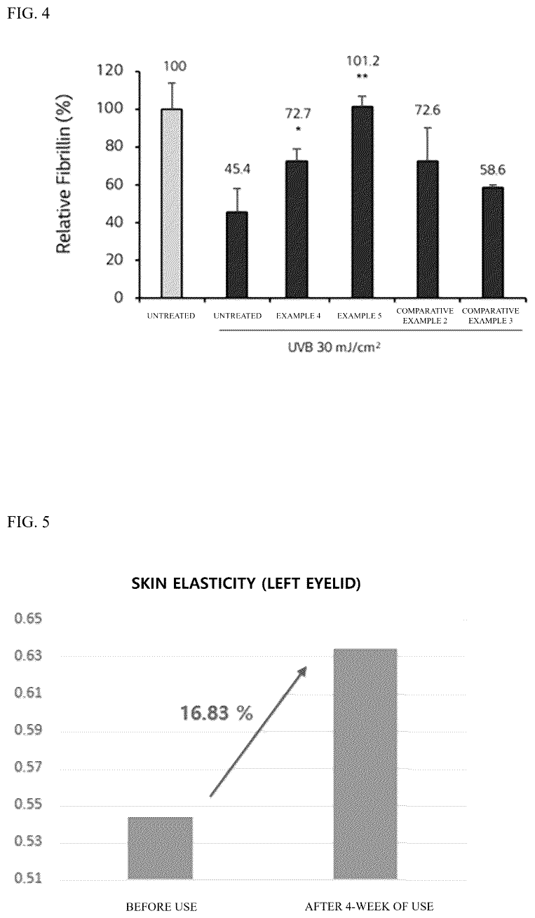

To measure the effect on the secretion of fibrillin, a glycoprotein essential for the formation of elastic fibers in skin connective tissue, human dermal fibroblasts (ATCC) were seeded at 2*10⁴ cells/well in 24-well plates and cultured overnight in a CO₂ incubator. To examine the effect of the solutions of Comparative Examples and Examples on fibrillin secretion under UV irradiation, one of the causes of aging, cells were irradiated with UVB at an intensity of 30 mJ/cm², and the solutions of Comparative Examples and Examples were added to the culture medium at their final contents and cultured in an incubator for 24 hours. After culture, the solutions and cells were washed with 1×PBS, and the cells were fixed by treating 4% paraformaldehyde for 10 minutes. After 0.1% Triton X-100 was treated for 10 minutes to allow antibodies to enter the cells, a blocking process was performed with a PBS solution containing 5% FBS for 30 minutes. An anti-fibrillin (Abcam ab53076) primary antibody was treated at a 1:250 ratio at 4° C. overnight, and then a goat anti-rabbit IgG (Abcam ab150077) secondary Ab was treated at a 1:1000 ratio for 1 hour in the dark to conduct the antibody-protein binding reaction. For nuclear staining, the cells were stained with DAPI and observed under a microscope. The amount of fibrillin protein, which appears as fluorescent green, was quantified using the open-source software Image J (FIGS. 3 and 4).

As shown in FIGS. 3 and 4, the elasticity protein fibrillin decreased about 54.6% under UVB irradiation conditions. Comparative Examples 2 and 3 did not show a significant increase in fibrillin synthesis compared to the untreated control after UVB irradiation, but Examples 4 and 5 showed increases of 27.3% and 55.8% in fibrillin synthesis, respectively. In particular, the solution of Example 5, which contained the complex plant extract, complex plant-derived glycoprotein extract, and two amino acids, demonstrated the highest increase in fibrillin synthesis, and the protein level was confirmed to be similar to that observed before UVB irradiation.

### Experimental Example 4: Formulation Stability and Sensory Evaluation

### Example 4-1: Preparation of Cosmetic Composition

The compositions were prepared according to the composition ratios (units: wt %) listed in Table 5 below by dissolving raw materials 2 and 3 in raw material 1, adding raw material 4 and uniformly dispersing the raw materials, and then adding and uniformly stirring raw material 5 in the dispersion and sequentially adding and uniformly stirring raw materials 6 to 8 to prepare Preparation Examples 1 to 4.

TABLE 5

| Raw | | Preparation | Preparation | Preparation | Preparation |
|---|---|---|---|---|---|
| material | Component | Example 1 | Example 2 | Example 3 | Example 4 |
| 1 | Purified | To 100 | To 100 | To 100 | To 100 |
| | water | | | | |
| 2 | Dipropylene | 5.0 | 5.0 | 5.0 | 5.0 |
| | glycol | | | | |
| 3 | Neutralizer | Appro- | Appro- | Appro- | Appro- |
| | | priate | priate | priate | priate |
| | | amount | amount | amount | amount |
| 4 | Hydrogenated | 1.5 | 1.5 | 1.5 | 1.5 |
| | polydecene | | | | |
| 5 | Carbomer | 0.2 | 0.2 | 0.2 | 0.2 |
| 6 | Preservative | Appro- | Appro- | Appro- | Appro- |
| | | priate | priate | priate | priate |
| | | amount | amount | amount | amount |
| 7 | Fragrance | Appro- | Appro- | Appro- | Appro- |
| | | priate | priate | priate | priate |
| | | amount | amount | amount | amount |
| 8 | Comparative | 1.0 | 5.0 | 1.0 | 1.0 |
| | Examples 2 | (Com- | (Com- | (Exam- | (Exam- |
| | and 3 or | parative | parative | ple 4) | ple 5) |
| | Examples 4 | Example | Example | | |
| | and 5 | 2) | 3) | | |

### Example 4-2: Evaluation of Composition Stability Through Off-Odor Development

The cosmetic compositions of Preparation Examples 1 to 4 were stored at 25° C. for 4 weeks, 4° C. for 4 weeks, and 45° C. for 4 weeks to check for changes in stability (development of off-odors or unpleasant odors), and results are shown in Table 6.

TABLE 6

| | Preparation | Preparation | Preparation | Preparation |
|---|---|---|---|---|
| | Example 1 | Example 2 | Example 3 | Example 4 |
| Room temperature | O | Δ | O | O |
| (25° C.) | | | | |
| Refrigerated (4° C.) | O | O | O | O |
| High temperature | O | X | O | O |
| (45° C.) | | | | |

<Evaluation criteria> No issues: O, Slight off-odor: Δ, Strong off-odor: X

Preparation Example 2, which contained 5% complex plant extract, showed slight to strong off-odors under room temperature and high temperature conditions for 4 weeks, indicating decreased stability of the cosmetic composition. Preparation Examples 1, 3, and 4 with extract contents similar to 1% complex plant extract maintained stability without unpleasant odor development even at a high temperature.

### Example 4-3: Sensory Evaluation

Sensory evaluation was conducted on 10 evaluators using the cosmetic compositions of Preparation Examples 1 to 4. After washing their left forearm with soap, the evaluators allowed their skin to adapt to a constant temperature/humidity environment (temperature of 22±2° C. and relative humidity of 45±5%) for 15 minutes. Afterward, the test samples were slowly applied to the skin and evaluated for 2 criteria, and the average values of the evaluation results are shown in Table 7 below.

TABLE 7

| | Preparation | Preparation | Preparation | Preparation |
|---|---|---|---|---|
| | Example 1 | Example 2 | Example 3 | Example 4 |
| Stickiness | 4.2 | 2.8 | 4.2 | 4.5 |
| Oiliness | 3.8 | 2.9 | 4.0 | 4.2 |

<Evaluation criteria> 5 points: Very good, 4 points: Good, 3 points: Average, 2 points: Poor, 1 point: Very poor

For Preparation Examples 1 and 2, as the content of the complex plant extract increased, the stickiness evaluation scores decreased due to increased content of high-viscosity, low-volatility alcohols in the extract. In contrast, Preparation Examples 3 and 4 had stickiness evaluation scores equal to or higher than Preparation Example 1.

A similar trend was observed for the oiliness criterion. For Preparation Examples 1 and 2, oiliness evaluation scores decreased as the extract content increased due to the refractive index effect of alcohols in the complex plant extract. However, Preparation Examples 3 and 4 had oiliness evaluation scores similar to or higher than Preparation Example 1. Therefore, it was confirmed that Preparation Examples 3 and 4 have a high level of satisfaction in terms of the feeling of use.

### Experimental Example 5: Evaluation of Elasticity Efficacy

A cosmetic (cream) was prepared by applying at least 1% of the mixed composition of Example 5. This was applied to the skin to evaluate its effect on improving skin elasticity. The specific evaluation conditions are presented in Table 8 below, and the evaluation results are shown in FIG. 5.

TABLE 8

| | 31 adult women aged 30 to 60 in |
|---|---|
| Test subject | international age |
| Test period | 4 weeks |
| Test site | Face (21 areas) |
| Test item | Skin elasticity |

As indicated in FIG. 5, when the mixed composition of the present invention was applied to the skin, it was confirmed that after 4 weeks of use, the skin elasticity improvement rate was maximally increased in the left eyelid among the tested 21 facial areas, with an improvement rate of 16.83%.

### Preparation Examples 5 and 6: Preparation of Cosmetic Composition

The compositions were prepared according to the composition ratios (units: wt %) listed in Tables 9 and 10 below by dissolving raw materials 2 and 3 in raw material 1, adding raw material 4 and uniformly dispersing the raw materials, and then adding and uniformly stirring raw material 5 in the dispersion and sequentially adding and uniformly stirring raw materials 6 to 8 to prepare the cosmetic compositions.

TABLE 9

| Raw material | Component | Preparation Example 5 |
|---|---|---|
| 1 | Purified water | To 100 |
| 2 | Glycerin | 1-5 |
| 3 | Neutralizer | Appropriate amount |
| 4 | Dimethicone | 1-5 |
| 5 | Carbomer | 0.1-0.3 |
| 6 | Preservative | Appropriate amount |
| 7 | Fragrance | Appropriate amount |
| 8 | Example 6 | 1.0 |

TABLE 10

| Raw material | Component | Preparation Example 6 |
|---|---|---|
| 1 | Purified water | To 100 |
| 2 | Dipropylene glycol | 2-6 |
| 3 | Neutralizer | Appropriate amount |
| 4 | Hydrogenated polydecene | 1.5 |
| 5 | Carbomer | 0.2 |
| 6 | Preservative | Appropriate amount |
| 7 | Fragrance | Appropriate amount |
| 8 | Example 6 | 5.0 |

### Experimental Example 6: Results of Promoting Expression of Elasticity-Related Factors COL1A1 and COL4A1 by Glycoprotein Plant Extract

To confirm the effect of the glycoprotein plant extract of the present invention on promoting the expression of elasticity-related factors, fibroblasts were treated with varying concentrations of the glycoprotein plant extract to evaluate whether it promotes COL1A1 and COL4A1 expression.

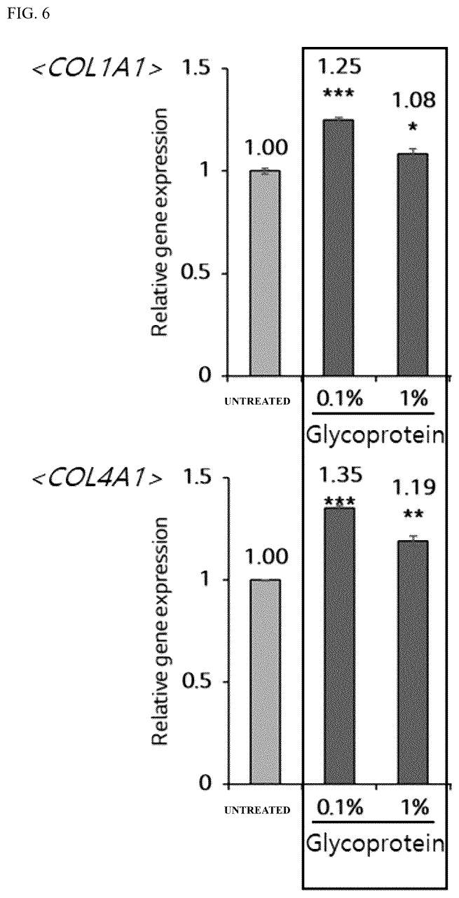

Specifically, human dermal fibroblasts (ATCC) were seeded at a density of 2*10⁵ cells/well in a 6-well plate and cultured overnight in a CO₂ incubator. The glycoprotein plant extract solution was then added to the culture medium at its final content and cultured in the incubator for an additional 24 hours. RNA was extracted according to the protocol using the RNA-spin Total RNA Extraction Kit (Intron Biotechnology, Korea), and real-time qPCR was performed using SYBR Green Primer to determine the expression levels of COL1A1 and COL4A1 induced by the glycoprotein plant extract solution (FIG. 6).

As shown in FIG. 6, the glycoprotein plant extract of the present invention promoted the expression of collagen synthesis factor by 108% to 135% and was confirmed to contribute to enhancing skin elasticity.

### Experimental Example 7: Results of Inhibiting Expression of Elasticity-Degrading Enzyme by Glycoprotein Plant Extract

To confirm the effect of the glycoprotein plant extract of the present invention on inhibiting the expression of elasticity-degrading factors, fibroblasts were treated with ultraviolet light to promote the expression of elasticity-degrading factors, and the inhibitory effect of the glycoprotein plant extract was evaluated.

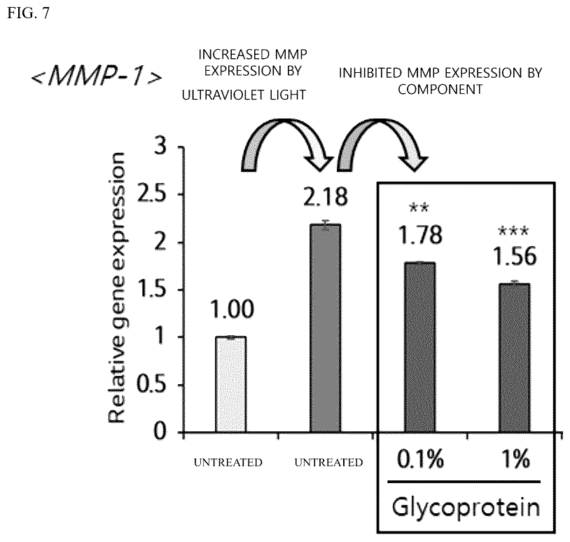

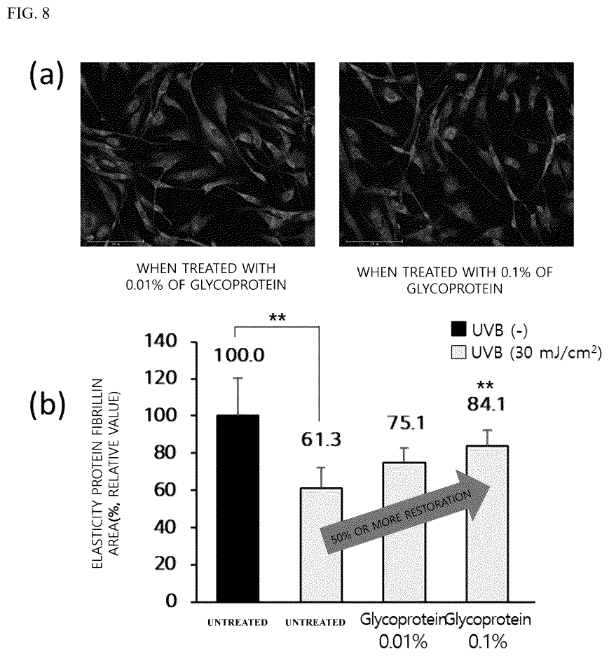

Specifically, human dermal fibroblasts (ATCC) were seeded at a density of 2*10⁵ cells/well in a 6-well plate and cultured overnight in a CO₂ incubator. After irradiating cells with UVB at an intensity of 20 mJ/cm², the glycoprotein plant extract solution was added to the culture medium at its final content and cultured in the incubator for an additional 24 hours. RNA was extracted according to the protocol using the RNA-spin Total RNAExtraction Kit (Intron Biotechnology, Korea), and real-time qPCR was performed using SYBR Green Primer to determine the expression levels of MMP-1 induced by the glycoprotein plant extract solution (FIG. 7).

As shown in FIG. 7, it was confirmed that treatment with the glycoprotein plant extract of the present invention at concentrations of 0.1 to 1% inhibited MMP-1 expression induced by aging by about 18% to 28%, thereby preventing loss of elasticity.

### Experimental Example 8: Results of Fibrillin Elasticity Factor Production by Glycoprotein Plant Extract

To confirm the effect of the glycoprotein plant extract of the present invention on the production of the elasticity factor fibrillin, fibroblasts were irradiated with ultraviolet light and then treated with the glycoprotein plant extract to assess the increase in fibrillin production.

Specifically, human dermal fibroblasts (ATCC) were seeded at a density of 2*10⁴ cells/well in a 24-well plate and cultured in a CO₂ incubator overnight. To examine the effect of the glycoprotein plant extract solution on fibrillin secretion under UV irradiation, which is one of the causes of aging, cells were irradiated with UVB at an intensity of 30 mJ/cm², and the glycoprotein plant extract solution was added to the culture medium at its final content and cultured in the incubator for 24 hours. After culture, the solution and cells were washed with 1×PBS, and the cells were fixed by treating 4% paraformaldehyde for 10 minutes. After 0.1% Triton X-100 was treated for 10 minutes to allow antibodies to enter the cells, a blocking process was performed with a PBS solution containing 5% FBS for 30 minutes. An anti-fibrillin (Abcam ab53076) primary antibody was treated at a 1:250 ratio at 4° C. overnight, and then a goat anti-rabbit IgG (Abcam ab150077) secondary Ab was treated at a 1:1000 ratio for 1 hour in the dark to conduct the antibody-protein binding reaction. For nuclear staining, the cells were stained with DAPI and observed under a microscope. The amount of fibrillin protein, which appears as fluorescent green, was quantified using the open-source software Image J (FIG. 8).

As shown in FIG. 8, it was confirmed that treatment with 0.1% of the glycoprotein plant extract of the present invention was effective in producing elasticity factors by restoring 50% or more of the reduction in fibrillin caused by aging induction.

### Experimental Example 9: Results of Promoting Expression of Hyaluronic Acid Synthesis Factor by Glycoprotein Plant Extract

To confirm the effect of the glycoprotein plant extract of the present invention on promoting the expression of hyaluronic acid synthesis factors, keratinocytes were treated with the glycoprotein plant extract, and the results of hyaluronic acid synthesis were evaluated.

Specifically, human adult low calcium high temperature epithelial keratinocytes (AddexBio Technologies) were seeded at a density of 2*10⁵ cells/well in a 6-well plate and cultured in a CO₂ incubator overnight. After irradiating cells with UVB at an intensity of 20 mJ/cm², the glycoprotein plant extract solution was added to the culture medium at its final content and cultured in the incubator for an additional 24 hours. RNA was extracted according to the protocol using the RNA-spin Total RNA Extraction Kit (Intron Biotechnology, Korea), and real-time qPCR was performed using SYBR Green Primer to determine the expression levels of HAS2 induced by the glycoprotein plant extract solution (FIG. 9).

As shown in FIG. 9, it was confirmed that treatment with 0.01 to 0.1% of the glycoprotein plant extract increased the expression of the hyaluronic acid synthesis factor HAS2 by up to 313%, thereby enhancing skin elasticity and exhibiting a moisturizing effect by promoting the production of hyaluronic acid, which has excellent moisturizing properties due to its strong water-binding capacity.

### Experimental Example 10: Results of Differentiation of Adipose-Derived Stem Cells and Lipid Accumulation by Glycoprotein Plant Extract

The differentiation of adipose-derived stem cells and the adipogenesis of the glycoprotein plant extract of the present invention were confirmed.

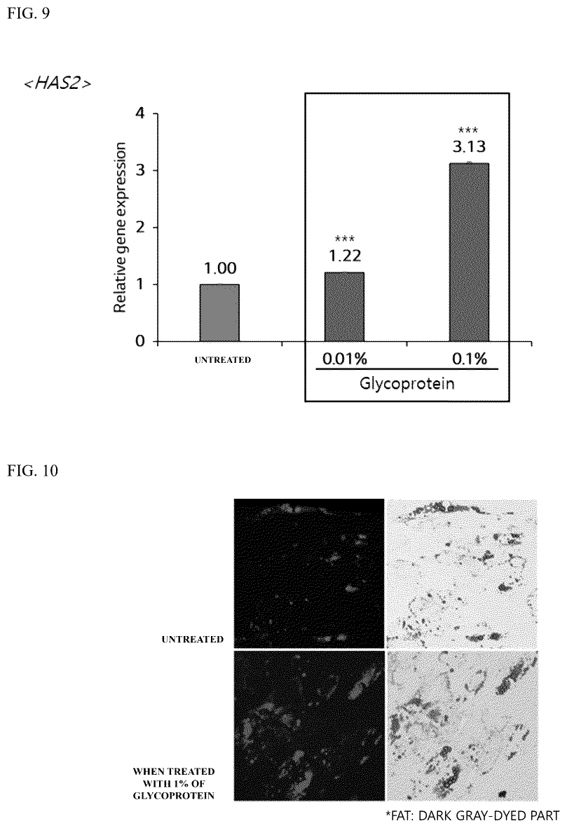

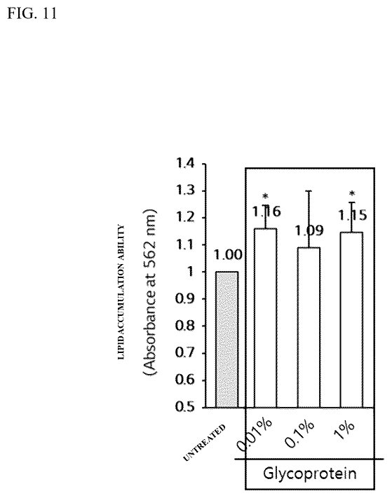

Specifically, adipose-derived stem cells (Cellbio) were seeded at a density of 5*10⁴ cells/well in a 24-well plate and cultured overnight in a CO₂ incubator. The glycoprotein plant extract solution was then added to the differentiation culture medium for adipose-derived stem cells at its final content and cultured in the incubator for an additional 24 hours. The differentiation culture medium for adipose-derived stem cells was prepared by adding the differentiation composition (PGM-2 Bullet kit, Lonza) consisting of fetal bovine serum, L-glutamine, recombinant human insulin (0.1%), dexamethasone, IBMX, indomethacin, and GA-1000 to preadipocyte basal medium-2 (PBM-2, Lonza). The culture medium was replaced every 3-4 days over 10-14 days during cultivation. After differentiation was completed, the cell samples were washed with DPBS and fixed with 10% formalin. The samples were washed with distilled water and treated with 60% isopropanol for 2 minutes. After removing the supernatant, the samples were allowed to react with 60% oil red O (DW 40%) for 20 minutes. After removing the supernatant again, the samples were washed with distilled water and observed using a microscope (EVOS™ M7000, Thermo Fisher Scientific). To elute pigments, 100% isopropanol was allowed to react for 10 minutes and collected, and then the absorbance was measured at 515 nm (FIGS. 10 and 11).

As shown in FIGS. 10 and 11, it was confirmed that when treated with 0.01 to 0.1% of the glycoprotein plant extract, the lipid accumulation ability was improved to a level of about 15%. Therefore, it can be seen that the glycoprotein plant extract of the present invention has an effect on enhancing elasticity through improving fat volume.

Based on the results from Experimental Examples 1 to 10 of the present invention, it was confirmed that the cosmetic composition of the present invention effectively improves the feeling of use of plant and herbal extracts, solves problems of odor and formulation stability, and is effective for enhancing skin elasticity by additionally combining glycoproteins and amino acids compared to simply including a higher content of complex plant extract as an active ingredient.

The cosmetic composition of the present invention solves the problems of odor and formulation stability of plant and herbal medicine extracts, and has the effects of slowing skin aging and reducing wrinkles.

From the above description, those skilled in the art to which the present invention pertains will understand that the present invention can be implemented in other specific forms without changing its technical idea or essential features. Therefore, the embodiments described above should be understood in all respects as illustrative and not restrictive. The scope of the present invention should be interpreted to include all changes or modified forms derived from the meaning and the scope of the claims described below, rather than being limited by the detailed description above.

1. A cosmetic composition comprising a glycoprotein plant extract of Angelica gigas root, Cornus officinalis fruit, Thymus vulgaris, Ganoderma lucidum, and Asparagus cochinchinensis root as an active ingredient.

2. The composition of claim 1, wherein the glycoprotein is obtained by treating the plants with one or more proteases.

3. The composition of claim 1, wherein the glycoprotein plant extract contains a sum of aspartic acid, arginine, and glutamic acid that is 30% or more of the total amino acids constituting the glycoprotein.

4. The composition of claim 1, further comprising an amino acid.

5. The composition of claim 4, wherein the amino acid includes one or more selected from the group consisting of arginine, histidine, lysine, aspartic acid, serine, threonine, asparagine, glutamine, tyrosine, alanine, isoleucine, leucine, valine, phenylalanine, methionine, tryptophan, glycine, proline, cysteine, and glutamic acid.

6. The composition of claim 1, wherein the glycoprotein plant extract is included in an amount of 0.0001 to 10 parts by weight based on 100 parts by weight of the composition.

7. The composition of claim 4, wherein the amino acid is included in an amount of 0.00001 to 10 parts by weight based on 100 parts by weight of the composition.

8. The composition of claim 1, further comprising a plant extract of Angelica gigas root, Cornus officinalis fruit, Thymus vulgaris, Ganoderma lucidum, and Asparagus cochinchinensis root as an active ingredient.

9. The composition of claim 8, wherein the plant extract is included in an amount of 0.01 to 30 parts by weight based on 100 parts by weight of the composition.
